(19) United States
(12) Patent Application Publication
(10) Pub. No.: US 2012/0082995 A1
(43) Pub. Date: Apr. 5, 2012

(54) METHOD FOR QUANTITATIVE PCR AMPLIFICATION OF DEOXYRIBONUCLEIC ACIDS FROM A SAMPLE CONTAINING PCR INHIBITORS

(76) Inventor: Jaakko Kurkela, Espoo (FI)

(21) Appl. No.: 13/377,683

(22) PCT Filed: Jun. 28, 2010
(86) PCT No.: PCT/FI10/50554

§ 371 Date: Dec. 12, 2011

(30) Foreign Application Priority Data
Jun. 26, 2009 (FI) 20095729

(51) Int. Cl. C12Q 1/68

(52) U.S. Cl. 435/6.12

(57) ABSTRACT

The present invention is directed to a method for quantitative PCR amplification of deoxyribonucleic acids (DNA) from a sample containing PCR inhibitors such as biological, clinical or environmental samples. In the method of the invention an inhibitor-tolerant DNA polymerase is used in a pre-amplification step to increase the copy number of DNA from these samples. In the pre-amplification step, the PCR reaction preferably comprises at least the same amount of effective PCR inhibitors as a reaction with 1% (v/v) human blood. The pre-amplified sample is subsequently diluted in order to dilute inhibitory substances remaining in the sample and thus rendering possible to use an aliquot of the diluted sample in quantitative PCR, which is very sensitive for these inhibitors.

The present invention is directed to a method for quantitative PCR amplification of deoxyribonucleic acids (DNA) from a sample containing PCR inhibitors such as biological, clinical or environmental samples. In the method of the invention an inhibitor-tolerant DNA polymerase is used in a pre-amplification step to increase the copy number of DNA from these samples. In the pre-amplification step, the PCR reaction preferably comprises at least the same amount of effective PCR inhibitors as a reaction with 1% (v/v) human blood. The pre-amplified sample is subsequently diluted in order to dilute inhibitory substances remaining in the sample and thus rendering possible to use an aliquot of the diluted sample in quantitative PCR (qPCR).

### BACKGROUND OF THE INVENTION

Quantitative real-time PCR (qPCR) is a method for DNA amplification in which fluorescent dyes are used to detect the amount of PCR product after each PCR cycle. (Higuchi et al., 1992). The qPCR method has become the tool of choice for many scientists because of method's dynamic range, accuracy, high sensitivity, specificity and speed. The method is highly suitable, e.g., for obtaining data on viral or bacterial load from infection patients, to monitor cancer, gene expression studies and to examine the genetic basis for individual variation in drug response. However, the use of qPCR is restricted to DNA samples which are highly purified or diluted, since the method is very sensitive to PCR inhibition.

Biological and environmental samples generally contains high concentrations of natural PCR inhibitors, which significantly hinder the activity of DNA polymerases or other factors indispensable for the sensitivity and efficiency of a PCR reaction (reviewed by Rådström et al., 2003). Inhibitory compounds can also generate background fluorescence or stain the sample with improper color which interfere with fluorescence detection in qPCR. PCR inhibitors can also originate from sample preparation, such as residual phenol from DNA extractions.

It is known that DNA polymerases vary in their tolerance of PCR inhibitors. For example, DNA polymerases widely used in real-time and quantitative PCR, such as AmpliTaq Gold® and Taq®, are completely inactive in the presence of 0.004% (v/v) human blood, whereas some novel modified DNA polymerases, such as Phusion® DNA polymerase, can be used with samples containing even 40% (v/v) blood. Unfortunately, the use of many of these modified DNA polymerases is limited in qPCR reactions, since they lack the 5′ exonuclease activity essential for many qPCR methods, like TaqMan® method (see U.S. Pat. No. 5,804,375).

In the prior art, the problem of PCR inhibition originating from DNA samples is solved in various ways, particularly by sample dilution and sample purification. Also pre-amplification steps have been introduced in several PCR based assays. However, pre-amplification is typically used to increase sample material to enable more assays to be done with limiting sample amount.

Gonzales et al., 2005, disclose a multiple displacement amplification, wherein DNA from environmental sample is pre-amplified with Φ29 DNA polymerase and random hexamer primers to increase the amount of DNA in the sample and simultaneously dilute inhibitory substances. This step is followed by PCR with target-specific primers. However, the pre-amplification step is performed with a reaction containing a diluted aliquot of lysed cells from the environmental sample. It seems that the dilution rate is significantly higher than in the method of the present invention, e.g., the reaction contains much less than 1% original environmental sample. Therefore, the pre-amplification step of Gonzales et al. is not dependent on the use of inhibitor-tolerant DNA polymerase. Furthermore, the multiple displacement amplification with Φ29 DNA polymerase may not be a proper pre-amplification step preceding qPCR, since it is very likely that different parts of the DNA in the sample is amplified with different efficiency.

A real-time PCR for SARS-coronavirus with target-gene pre-amplification is disclosed in Lau et al., 2003. However, in this method cDNA templates are isolated and purified from clinical samples before pre-amplification, and thus the problem of PCR inhibition originating directly from clinical samples is not addressed in the paper. Indeed, the aim of the authors is to increase sensitivity of real-time PCR not to lower the level of PCR inhibitors in the sample.

Doherty et al., 2002, disclose a quantitative assay for HIV with high sensitivity. In this assay, a DNA sample from a lysed IS cell line is diluted so that it is possible to use a regular DNA polymerase, such as Platinum Taq DNA polymerase™, for pre-amplification.

In Qi et al., 2001 a pre-amplification step was used to achieve higher signal-to-noise ratio in UV-detection system. However, DNA samples for PCR reactions were prepared using a standard DNA purification method.

Use of a pre-amplification step is also disclosed in the following patent publications: WO 2008157501, U.S. Pat. No. 7,118,868, WO 06099164, WO 06102352, US 20060008807, and U.S. Pat. No. 6,410,223, but in all documents the aim of pre-amplification is not related to the reduction of inhibitors in PCR reactions. Moreover, the components of the pre-amplification steps of prior art are different from the components used in the method of the present invention.

### BRIEF DESCRIPTIONS OF DRAWINGS

FIG. 1. qPCR results of a blood sample pre-amplified with Phusion DNA polymerase.

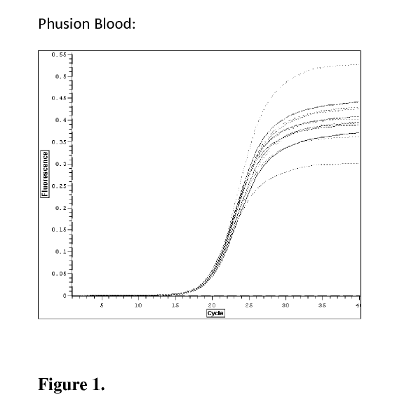

FIG. 2. qPCR results of a blood sample pre-amplified with DyNAzyme II DNA polymerase.

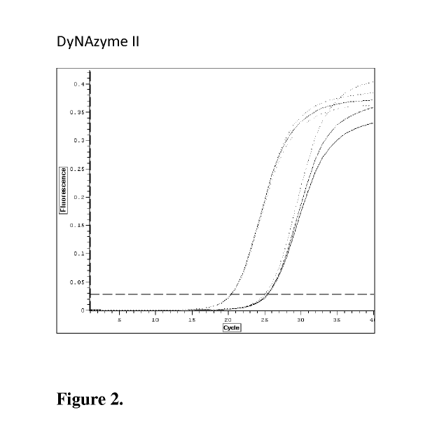

FIG. 3. qPCR results of a blood sample pre-amplified with AmpliTaq Gold DNA polymerase.

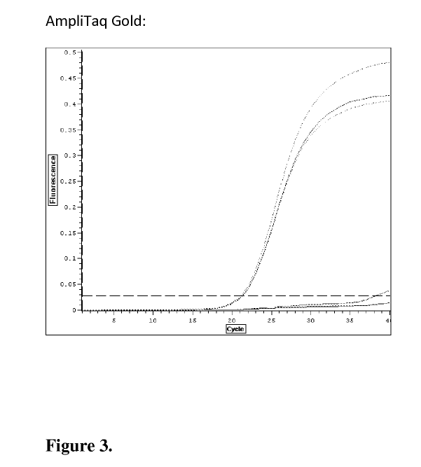

### DETAILED DESCRIPTION OF THE INVENTION

The present invention is directed to a method for quantitative PCR amplification of deoxyribonucleic acids from a sample containing PCR inhibitors, the method comprising the steps of:

a) preparing a PCR reaction comprising an aliquot of said sample containing PCR inhibitors, an inhibitor-tolerant DNA polymerase and suitable reagents needed for a PCR reaction with said DNA polymerase, wherein the inhibitory effect of said PCR reaction is at least the same as in the reaction containing 1% human blood;
b) subjecting the reaction of step a) to at least 5 cycles of denaturation, annealing and extension;
c) diluting the amplified reaction of step b) according to a ratio of at least 1:10;
d) performing quantitative PCR amplification and preferably an analysis to a reaction containing an aliquot from the diluted reaction of step c).

The present method is specifically directed to facilitate quantitative analysis of DNA samples containing PCR inhibitors in such an amount that standard DNA polymerases used in qPCR are inhibited. In the context of the present invention the expression “a sample containing PCR inhibitors” means a sample the DNA of which cannot be quantitatively amplified with a standard DNA polymerases but is amplified with an inhibitor-tolerant DNA polymerase such as Phusion™ DNA polymerase. Preferably, these samples are biological, clinical, food, or environmental samples. A good example of a clinical sample suitable for the present invention is a blood sample, particularly human blood sample, serum sample or a sample containing a fraction of blood. The present method is thus particularly suitable for assaying, e.g., sepsis-causing bacteria, since the method provides a quick and efficient means to specifically detect microbial DNA in the blood of a patient. This facilitates the decision of a physician regarding the selection of correct antibiotic for the patient suffering from sepsis.

Other preferred sample for the method of the invention is a milk sample. It is also in the scope of the invention to use materials from cell cultures, biopsies and tissue samples in the method. It should be noted that in the preferred embodiment of the invention, said sample containing PCR inhibitors is an untreated sample directly obtained from the donor, cell culture or environment, as it is advantageous to add the sample material directly to the PCR reaction of step a) without previous steps of sample preparation, such as DNA extraction.

The PCR reaction for step a) is preferably prepared so that it contains at least 1% (v/v) said sample containing PCR inhibitors, e.g., at least 1% (v/v) blood. If the final volume of the PCR reaction is 50 μl, then it contains at least 0.5 μl said sample. Advantageously, the PCR reaction can contain even higher amounts of said sample, e.g., 2 to 40% (v/v) reactions are possible. Preferred total volumes of the reactions are 20 μl and 50 μl and preferred proportions of said sample in those reactions are 0.5-8 μl and 1-20 μl, respectively. The sample containing inhibitors can also be a solid sample, such as a blood spot (for example on an FTA® card (Whatman)) or a tissue sample, which may be added directly to the pre-amplification reaction of step a). Thus, any sample that have at least the same inhibitory effect to inhibitor-sensitive DNA polymerases as a sample containing 1% human blood is a sample containing PCR inhibitors as defined in the present invention (see Example 1 below).

An inhibitor-tolerant DNA polymerase is a DNA polymerase enzyme which retains its typical level of amplification activity in the presence of 1% (v/v) human blood. In the Example 1 of the Experimental Section, a specific test for determining the activity of DNA polymerases in the presence of PCR inhibitors is disclosed. At least the following are inhibitor-tolerant DNA polymerases as defined herein: Phusion® (Finnzymes, see WO 01/92501), Hemo KlenTaq® (New England Biolabs), and Kapa® Blood (Kapa Biosystems) DNA polymerases.

Suitable reagents for a PCR reaction in the method of the invention can be selected from the group consisting of: primers, probes, dyes, labels, nucleotides, salts, buffering agents, various additives and PCR enhancers. Preferably, the reactions with inhibitor-tolerant DNA polymerases are prepared to the buffer recommended by the manufacturer. Primers for the pre-amplification of step a) and the qPCR reaction of step b) can be the same, i.e. primers which are specific for the amplicon of interest. However, the approach of “nested PCR” can also be used, wherein the amplified DNA stretch in step a) is longer than the amplicon targeted in qPCR of step d). In the qPCR reaction, all conventional reagents and materials, such as target specific labeled probes and double-stranded DNA binding dyes (e.g. SYBR™ Green), can be used.

The pre-amplification reaction may also be a multiplex pre-amplification reaction, i.e. containing at least two amplicons amplified simultaneously in the same reaction vial.

In the present method, the reaction of step a) is subjected to at least 5 cycles, preferably 8-12 cycles, of denaturation, annealing and extension in step b). The aim of this step is to pre-amplify the DNA template so that the amount of target DNA is preferably increased 50 to 10000 times. This renders possible dilution of inhibiting substances in the pre-amplified reaction without decreasing the number of DNA template to a level where the subsequent qPCR reaction does not contain statistically significant number of the template per reaction. Further, it is advantageous to use less than 15 cycles of pre-amplification, since quantitative nature of the pre-amplification reaction might be otherwise compromised, if more cycles are executed. Moreover, higher template copy number in the finalized reaction increases the risk of laboratory contamination as the tubes used for pre-amplification need to be opened while continuing the method with the qPCR step.

The duration and temperature of each phase in the pre-amplification step, i.e. denaturation, annealing and extension, may vary based on the DNA polymerase and sample material used. However, a skilled person of the art can easily optimize any PCR reaction and thus the present invention is not limited to any certain cycle pattern used in the art. However, usually the initial denaturation in the first cycle of amplification is longer than the denaturation steps in the subsequent cycles (i.e. “hot start”). Further, annealing and extension may be combined into a one phase (i.e. 2-step-PCR). Examples of suitable PCR reactions are presented in the Experimental Section below.

The pre-amplification step of the method is followed by dilution of the amplified reaction. The reaction is diluted according to a ratio of at least 1:10. Preferred dilution rates are between 1:50 and 1:5000. Most preferred dilution rates are between 1:500 and 1:1000. For clarity, it is noted that the dilution rate 1:10 relates to a dilution, wherein one part of the amplified reaction is mixed with nine parts of suitable buffer. Accordingly, dilution rate 1:500 relates to a dilution consisting of one part of the amplified reaction and 499 parts of suitable buffer.

Tables 1-2 below clarify the relation of amplification factor and feasible dilution range after preamplification.

Table 1 shows how many cycles are needed to achieve different amplification factors with different amplification efficiencies. For example 1000 fold dilution after 10 cycles of 100% efficient amplification yields approximately the same concentration of the amplified target that was present in the original sample. If higher dilution is needed, for example if the preamplification reaction contains a lot of inhibiting material (inhibitory to the following qPCR—not preamplification), more cycles may be needed to end up with enough target in the diluted sample. With efficient amplification high enough amplification is achieved with even few cycles especially if there is no need for high dilution due to small inhibition in the sample.

TABLE 1

| Relationship between numberof cycles and | |
|---|---|
| amplification factor with 100% and 80% efficiencies. | |
| Number | Amplification factor |
| of cycles | with 100% efficiency |
| 5 | 32 |
| 6 | 64 |
| 7 | 128 |
| 8 | 256 |
| 9 | 512 |
| 10 | 1024 |
| 11 | 2048 |
| 12 | 4096 |
| 15 | 32768 |
| 20 | 1048576 |
| Number | Amplification factor |
| of cycles | with 80% efficiency |
| 5 | 19 |
| 6 | 34 |
| 7 | 61 |
| 8 | 110 |
| 9 | 198 |
| 10 | 357 |
| 11 | 643 |
| 12 | 1157 |
| 15 | 6747 |
| 20 | 127482 |

If amplification efficiency is somewhat lower amplification factors are not as great. High enough amplification can still be achieved in less than 20 cycles even if required dilution is high due to high concentration of original inhibition in the sample.

TABLE 2
Blood concentration equivalent in percentage after dilution with different original
sample concentrations (in percentage) in preamplification step.

| Dilution | Original sample concentration in preamplification as percentage. | | | | | | | | | |
|---|---|---|---|---|---|---|---|---|---|---|
| factor | 0.025 | 0.05 | 0.1 | 0.25 | 0.5 | 1 | 2 | 4 | 8 | 16 |
| 10 | 0.0025 | 0.0050 | 0.0100 | 0.0250 | 0.0500 | 0.1000 | 0.2000 | 0.4000 | 0.8000 | 1.6000 |
| 20 | 0.0013 | 0.0025 | 0.0050 | 0.0125 | 0.0250 | 0.0500 | 0.1000 | 0.2000 | 0.4000 | 0.8000 |
| 30 | 0.0008 | 0.0017 | 0.0033 | 0.0083 | 0.0167 | 0.0333 | 0.0667 | 0.1333 | 0.2667 | 0.5333 |
| 40 | 0.0006 | 0.0013 | 0.0025 | 0.0063 | 0.0125 | 0.0250 | 0.0500 | 0.1000 | 0.2000 | 0.4000 |
| 50 | 0.0005 | 0.0010 | 0.0020 | 0.0050 | 0.0100 | 0.0200 | 0.0400 | 0.0800 | 0.1600 | 0.3200 |
| 60 | 0.0004 | 0.0008 | 0.0017 | 0.0042 | 0.0083 | 0.0167 | 0.0333 | 0.0667 | 0.1333 | 0.2667 |
| 70 | 0.0004 | 0.0007 | 0.0014 | 0.0036 | 0.0071 | 0.0143 | 0.0286 | 0.0571 | 0.1143 | 0.2286 |
| 80 | 0.0003 | 0.0006 | 0.0013 | 0.0031 | 0.0063 | 0.0125 | 0.0250 | 0.0500 | 0.1000 | 0.2000 |
| 90 | 0.0003 | 0.0006 | 0.0011 | 0.0028 | 0.0056 | 0.0111 | 0.0222 | 0.0444 | 0.0889 | 0.1778 |
| 100 | 0.0003 | 0.0005 | 0.0010 | 0.0025 | 0.0050 | 0.0100 | 0.0200 | 0.0400 | 0.0800 | 0.1600 |
| 150 | 0.0002 | 0.0003 | 0.0007 | 0.0017 | 0.0033 | 0.0067 | 0.0133 | 0.0267 | 0.0533 | 0.1067 |
| 200 | 0.0001 | 0.0003 | 0.0005 | 0.0013 | 0.0025 | 0.0050 | 0.0100 | 0.0200 | 0.0400 | 0.0800 |
| 250 | 0.0001 | 0.0002 | 0.0004 | 0.0010 | 0.0020 | 0.0040 | 0.0080 | 0.0160 | 0.0320 | 0.0640 |
| 300 | 0.0001 | 0.0002 | 0.0003 | 0.0008 | 0.0017 | 0.0033 | 0.0067 | 0.0133 | 0.0267 | 0.0533 |
| 350 | 0.0001 | 0.0001 | 0.0003 | 0.0007 | 0.0014 | 0.0029 | 0.0057 | 0.0114 | 0.0229 | 0.0457 |
| 400 | 0.0001 | 0.0001 | 0.0003 | 0.0006 | 0.0013 | 0.0025 | 0.0050 | 0.0100 | 0.0200 | 0.0400 |
| 450 | 0.0001 | 0.0001 | 0.0002 | 0.0006 | 0.0011 | 0.0022 | 0.0044 | 0.0089 | 0.0178 | 0.0356 |
| 500 | 0.0001 | 0.0001 | 0.0002 | 0.0005 | 0.0010 | 0.0020 | 0.0040 | 0.0080 | 0.0160 | 0.0320 |
| 600 | 0.0000 | 0.0001 | 0.0002 | 0.0004 | 0.0008 | 0.0017 | 0.0033 | 0.0067 | 0.0133 | 0.0267 |
| 700 | 0.0000 | 0.0001 | 0.0001 | 0.0004 | 0.0007 | 0.0014 | 0.0029 | 0.0057 | 0.0114 | 0.0229 |
| 800 | 0.0000 | 0.0001 | 0.0001 | 0.0003 | 0.0006 | 0.0013 | 0.0025 | 0.0050 | 0.0100 | 0.0200 |
| 900 | 0.0000 | 0.0001 | 0.0001 | 0.0003 | 0.0006 | 0.0011 | 0.0022 | 0.0044 | 0.0089 | 0.0178 |
| 1000 | 0.0000 | 0.0001 | 0.0001 | 0.0003 | 0.0005 | 0.0010 | 0.0020 | 0.0040 | 0.0080 | 0.0160 |
| 1500 | 0.0000 | 0.0000 | 0.0001 | 0.0002 | 0.0003 | 0.0007 | 0.0013 | 0.0027 | 0.0053 | 0.0107 |
| 2000 | 0.0000 | 0.0000 | 0.0001 | 0.0001 | 0.0003 | 0.0005 | 0.0010 | 0.0020 | 0.0040 | 0.0080 |
| 4000 | 0.0000 | 0.0000 | 0.0000 | 0.0001 | 0.0001 | 0.0003 | 0.0005 | 0.0010 | 0.0020 | 0.0040 |
| 10000 | 0.0000 | 0.0000 | 0.0000 | 0.0000 | 0.0001 | 0.0001 | 0.0002 | 0.0004 | 0.0008 | 0.0016 |
| 20000 | 0.0000 | 0.0000 | 0.0000 | 0.0000 | 0.0000 | 0.0001 | 0.0001 | 0.0002 | 0.0004 | 0.0008 |

Dilution required for preamplified sample in qPCR setup depends on amount of inhibition in the sample i.e. concentration of the sample and the inhibitor tolerance of the qPCR step.

Table 2 represents calculations of the resulting amount of the original sample in percentage after different dilutions. Values below 0.004% are underlined as this is generally the concentration above which normal Taq is inhibited.

The quantitative PCR of step d) is preferably performed as instructed in the prior art (see e.g. Bustin S. (ed.) (2004) A-Z of Quantitative PCR. IUL Biotechnology Series, International University Line, La Jolla, Calif., USA). The volume of an aliquot from the diluted reaction of step c), which is used for qPCR, is preferably 1 to 20 μl. The total volume of the qPCR reaction is preferably 50 μl, most preferably 20-50 μl, alternatively 1-20 μl. Step d) of the method can also be performed in the form of a microarray.

One aspect of the invention is to enable qPCR analysis in small volume for samples that have high inhibition. Recent qPCR instrumentation include models that are capable of performing qPCR in less than 1 μl volumes. In such system the amount of sample input volume is limited and in most cases some pre amplification is needed anyway. However none of current preamplification systems are capable of amplifying samples with significant inhibition. Thus the current invention is especially well suited to be used with these types of systems. For example if qPCR is performed in 50 nl instead of more typical 500 μl, a 1000 fold preamplification is needed to ensure same amount of target molecules for detection. If the sample contains significant inhibition, normal preamplification systems cannot perform unless the sample is significantly diluted before preamplification. However in most cases dilution prior to preamplification is not feasible as many targets are present in low concentrations and may be totally lost if sample is diluted too much. According to one embodiment of the invention, the total volume of the qPCR reaction is preferably 1 μl and most preferably 1 μl-50 nl.

The publications and other materials used herein to illuminate the background of the invention, and in particular, to provide additional details with respect to its practice, are incorporated herein by reference. The present invention is further described in the following examples, which are not intended to limit the scope of the invention.

### EXPERIMENTAL SECTION

### Example 1

### Blood Resistance PCR Assay

This assay determines whether a DNA polymerase reaction is blood resistant or not according to the following criteria. In a blood-resistant polymerase reaction DNA template should be amplified in the presence of 1% blood in 10 PCR cycles so that when this amplified PCR product is used as a template in a qPCR assay and compared to similar amplification without blood, the Ct difference (deltaCt, ΔCt) between these reactions should be less than 4 cycles. If this criteria is met, the DNA polymerase is blood-resistant (i.e. an inhibitor-tolerant DNA polymerase). Accordingly, the ΔCt value of an inhibitor-sensitive DNA polymerase is 4 or more in this assay.

It should be noted that blood resistance may be achieved by selecting a suitable polymerase or by polymerase modification or by optimizing buffer condition or PCR protocol.

Example of Blood Resistance Assay

Polymerase should be used as suggested in its instructions or instructions given in a system to be evaluated. Two pre-amplification reactions are prepared for each polymerase both having 1 000 000 copies of lambda DNA as a template: one with 1% whole blood and one without added blood.

It should not matter whether blood is preserved with EDTA as an anticoagulant or not, as the amount of blood in a reaction is so small that inhibitory effect of the anticoagulant should be minimal.

Primers are 23C Fwd (5′AGGCCGGGTTATTCTTGTTCTCT 3′) and 23C Rev (5′ TTCTGCGGGTTATTGCTTCTTTC 3′).

Concentration as instructed in polymerase instructions or if not instructed then 500 nM.

PCR protocol as instructed or if not instructed then following:

Initial denaturation 95° C. 10 min

Cycles:

| | |
|---|---|
| 95° C. | 15 s |
| 62° C. | 30 s |
| 72° C. | 30 s |

Cycle repeated 10 times

After 10 cycles of amplification samples are taken from reactions, diluted 1:1000 and the dilution is used as a template for qPCR.

qPCR setup:

| | |
|---|---|
| 2× qPCR master mix (e.g. DyNAmo SYBR | 10 μl |
| Flash Master mix containing hot start version | |
| of a modified Tbr DNA polymerase, Finnzymes) | |
| Primer mix (23C Fwd and 23C Rev), 2 μM each | 5 ul |
| 1:1000 diluted pre-amplification reaction | 5 μl |

qPCR cycling:

Initial denaturation:

| | |
|---|---|
| 95° C. | 7 min |

Cycles

| | |
|---|---|
| 95° C. | 10 s |
| 60° C. | 30 s |

Repeated 40 times

Melting curve analysis as instructed by instrument manufacturer.

Data is analyzed as with generally accepted settings. For example base line determined from a cycle range that really represents a baseline level of fluorescence and threshold settings so that Ct values from analysis are not affected by noise due to too low threshold setting or extra variation due to too high setting. (In general suitable threshold setting is such that small change in threshold should not affect deltaCt significantly)

As an assay quality check no template control (NTC) should be run. Ct from a NTC reaction can be an indication of a contamination that could potentially interfere with blood resistance assessment. NTC Ct should be at least 3 cycles later than the Ct from other reaction to be considered insignificant. Melting curve analysis can be used as another quality check. Melting curve analysis should confirm that the products amplified from pre-amplification reactions with and without the presence of blood are the same. Also a qPCR reaction with pure lambda DNA as a template without pre-amplification can be run as a positive control and to identify the correct melting profile for the PCR product.

### Example 2

### Pre-Amplification Test with Different DNA Polymerases Following a Standard qPCR Amplification

Reaction conditions were as recommended in data sheets of the DNA polymerases without addition of MgCl₂ etc.

23C-amplicon of Lambda-DNA was amplified so that reactions contain +/−1% blood.

All reactions included 0.5 μM primers, except the reaction with Herculase II Fusion included 0.25 μM primers.

Control qPCR samples contained 0.001% blood.

| 20 × DNA + primer mix | 0.5 μM |
|---|---|
| 23C Fwd 100 μM | 5 |
| 23C Rev 100 μM | 5 |
| lambdaDNA 10\hat{ }6 copies/μl | 20 |
| H2O | 370 |
| | 500 |

A) Pre-Mixes and Cycles for Pre-Amplification Reactions:

| Phusion Blood Polymerase | | Cycles: | | | |
|---|---|---|---|---|---|
| Premix: | 1x | 98° C. | 5 min | | |
| 2 × Phusion Blood Buffer * | 25 | 98° C. | 1 s | | |
| DNA + primer-mix | 20 | 62° C. | 5 s | 10x | |
| Blood 1:4-dilution/H₂O | 2 | 72° C. | 15 s | | |
| (25%) | | | | | |
| Phusion Blood polymerase | 1/2 | 72° C. | 1 min | | |
| H₂O | 2/1 | 4° C. | | | |
| | 50 | | | | |
| Dynazyme II Polymerase | | | | | |
| 2 U/μl | | | Cycles: | | |
| Premix: | 1x | | 94° C. | 1 min | |
| Dynazyme II | 1 | | 94° C. | 15 s | |
| F-511 10 × Buffer | 5 | | 62° C. | 10 s | 10x |
| 10 mM dNTPs | 1 | → 200 μM each | 72° C. | 40 s | |
| DNA + primer-mix | 20 | | 72° C. | 5 min | |
| Blood 1:4-dilution/H₂O | 2 | | | | |
| (25%) | | | | | |
| H₂O | 21 | | | | |
| | 50 | | | | |
| AmpliTaq Gold (ABI) | | Cycles: | | | |
| Premix: | 1x | 95° C. | 10 min | | |
| 10 × Reaction Buffer Gold | 5 | 95° C. | 30 s | 10x | |
| 10 mM dNTPs | 1 | 60° C. | 30 s | | |
| AmpliTaq Gold 1:2-dilution | 0.5 | 72° C. | 10 min | | |
| DNA + primer-mix | 20 | 4° C. | | | |
| Blood/H₂O 1:4-dilution | 2 | | | | |
| (25%) | | | | | |
| H₂O | 21.5 | | | | |
| | 50 | | | | |

B) qPCR Amplification

5 μl of 1:1000-dilution of pre-amplified reaction was used as a template or 5 μl lambda DNA-dilution as a control amplified with DyNAmo™ Flash SYBR® Green qPCR Kit (Finnzymes) containing hot start version of a modified Tbr DNA polymerase.

| Premix: | 1× | 100× |
|---|---|---|
| DyNAmo ™ Flash SYBR ® | | |
| Green qPCR 2× mastermix | 10 | 1000 |
| 23C Fwd 25 μM | 0.4 | 40 |
| 23C Rev 25 μM | 0.4 | 40 |
| Template | 5 | — |
| H₂O | 4.2 | 420 |
| | 20 | 1500 |

Program:

| 1. | 95° C. | 7 min |
|---|---|---|
| 2. | 95° C. | 10 s |
| 3. | 60° C. | 15 s |
| 4. | Plate read | |
| 5. | go to line 2 for 39 more times | |
| 6. | 60° C. | 1 min |
| 7. | Melting curve 60 to 95° C., read every 0.5° C., hold 2 s | |
| 8. | 30° C. | 10 s |
| | END | |

### RESULTS

Melting Curve Analysis

| | delta Ct |
|---|---|
| Phusion Blood | 0.053333 |
| Dynazyme II | 4.853333 |
| AmpliTaq Gold | 17.06 |

According to the results, only Phusion Blood is inhibitor resistant according to the definition of the invention as only its ΔCt value is 4 or less in this assay.

The invention is not limited to the polymerases used in the examples. Polymerases suitable for pre-amplification step can be determined using the test described above in conditions recommended by the polymerase manufacturer.

### REFERENCES

Gonzalez J M, Portillo M C, and Saiz-Jimenez C; Multiple displacement amplification as a pre-polymerase chain reaction (pre-PCR) to process difficult to amplify samples and low copy number sequences from natural environments, Environmental Microbiology, 2005, 7(7):1024-1028.
Higuchi et al. (1992) Simultaneous amplification and detection of specific DNA sequences. Biotechnology (N Y) 10:413-417.
Lok Ting Lau, Yin-Wan Wendy Fung, Freda Pui-Fan Wong, Selma Sau-Wah Lin, Chen Ran Wang, Hui Li Li, Natalie Dillon, Richard A. Collins, John Siu-Lun Tam, Paul K. S. Chan, Chen G. Wang and Albert Cheung-Hoi Yu; A real-time PCR for SARS-coronavirus incorporating target gene pre-amplification; Biochemical and Biophysical Research Communications, 2003, 312:1290-1296.
Una O'Doherty, William J. Swiggard, Deepa Jeyakumar, David McGain, and Michael H. Malim; A Sensitive, Quantitative Assay for Human Immunodeficiency Virus Type 1 Integration, J. Virol. 2002; 76(21):10942-10950.
Xiaoquan Qi, Saleha Bakht, Katrien M. Devos, Mike D. Gale and Anne Osbourn; L-RCA (ligation-rolling circle amplification): a general method for genotyping of single nucleotide polymorphisms (SNPs), Nucleic Acids Research, 2001, Vol. 29, No. 22 e116.
Rådström P, Löfström C, Lövenklev M, Knutsson R, and Wolffs P; Strategies for Overcoming PCR inhibition, Chapter 12, in PCR Primer, 2nd edition. Cold Spring Harbor Laboratory Press, Cold Spring Harbor, N.Y., USA, 2003.

1. Method for quantitative PCR amplification of deoxyribonucleic acids from a sample containing PCR inhibitors, the method comprising the steps of:
a) preparing a PCR reaction comprising an aliquot of said sample containing PCR inhibitors, an inhibitor-tolerant DNA polymerase and suitable reagents needed for a PCR reaction with said DNA polymerase, wherein inhibitory effect of said PCR reaction is at least the same as in the reaction containing 1% human blood;
b) subjecting the reaction of step a) to at least 5 cycles of denaturation, annealing and extension;
c) diluting the amplified reaction of step b) according to a ratio of at least 1:10;
d) performing quantitative PCR amplification to a reaction containing an aliquot from the diluted reaction of step c).

2. The method according to claim 1, wherein said sample containing PCR inhibitors is a biological, clinical, food, or environmental sample.

3. The method according to claim 2, wherein said clinical sample is a blood sample.

4. The method according to claim 3, wherein the PCR reaction of step a) comprises at least 1% blood.

5. The method according to claim 1, wherein said suitable reagents for a PCR reaction are selected from the group of consisting of: primers, probes, dyes, labels, nucleotides, salts, buffering agents, and PCR enhancers.

6. The method according to claim 1, wherein the reaction of step a) is subjected to 8-12 cycles of denaturation, annealing and extension in step b).

7. The method according to claim 1, wherein the initial denaturation in the first cycle of step b) is longer than the denaturation steps in the subsequent cycles.

8. The method according to claim 1, wherein the amplified reaction of step b) is diluted according to a ratio between 1:500 and 1:1000.

9. The method according to claim 1, wherein the primers for the pre-amplification of step a) and the qPCR reaction of step b) are the same, i.e. primers which are specific for the amplicon of interest.

10. The method according to claim 1, wherein the primers for the pre-amplification of step a) and the qPCR reaction of step b) are designed based on principles of nested PCR.
